(19) United States
(12) Patent Application Publication
(10) Pub. No.: US 2018/0217051 A1
(43) Pub. Date: Aug. 2, 2018

(54) ACOUSTIC-OPTICAL IMAGING METHODS AND SYSTEMS

(71) Applicants: CENTRE NATIONAL DE LA RECHERCHE SCIENTIFIQUE - CNRS, Paris (FR); INSTITUT NATIONAL DE LA SANTÉ ET DE LA RECHERCHE MÉDICALE- INSERM, Paris (FR)

(72) Inventors: François Ramaz, Kremlin Bicetre (FR); Mickaël Tanter, Bagneux (FR); Jean-Baptiste Laudereau, Paris (FR); Jean-Luc Gennisson, Cergy (FR)

(73) Assignees: CENTRE NATIONAL DE LA RECHERCHE SCIENTIFIQUE - CNRS, Paris (FR); INSTITUT NATIONAL DE LA SANTÉ ET DE LA RECHERCHE MÉDICALE- INSERM, Paris (FR)

(21) Appl. No.: 15/579,400

(22) PCT Filed: Jun. 2, 2015
(86) PCT No.: PCT/FR2015/051448

§ 371 Date: Dec. 4, 2017

(51) Int. Cl. G01N 21/17; G01N 21/49

(52) U.S. Cl. CPC G01N 21/1702; G01N 21/1717; G01N 21/49; A61B 5/0097; G01N 2021/1787; G01N 2201/123; G01N 2021/1729

(57) ABSTRACT

The invention relates to an acoustic-optical imaging method and system of a zone for observing an environment. The system includes an acquisition device comprising, a network of transducers for generating a plurality of non-focussed sound waves, a light-emitting device for emitting an incident light wave and generating marked light waves comprising an acoustic-optical component that is shifted in frequency by the non-focussed sound waves, and a detector for acquiring measurement signals. The system also comprises a processing device for determining a light intensity in the observation zone from the measurement signals.

The present invention relates to acoustic-optical imaging methods and systems.

More particularly, the invention relates to an acoustic-optical imaging method for imaging an observation area of a medium. Such a method is intended to obtain information in a non-invasive manner about the optical properties of an observation area located deep within a medium, such as biological tissue. For example, the optical properties may be a color, an absorption, or a structure of the biological tissue of the observation area. The observation area is for example located several millimeters or centimeters deep within an object, for example within a body, an organ, or an object.

Such methods are known, in which an ultrasonic acoustic wave focused on a focal spot in the observation area is generated in the observation area of the medium, and a light wave is simultaneously emitted in this same area. Information is then obtained by detecting a signal related to the link between the light wave and the acoustic vibration in the medium. Indeed, when an ultrasonic wave of acoustic frequency fa travels through a scattering medium (for example biological or other tissue), it causes a periodic displacement of the scatterers and a periodic modulation of the refractive index of the medium. If an incident light wave, in particular a laser wave, of incident frequency fi is scattered by the medium, the movement of the scatterers and the modulation of the refractive index of the medium generate a marked light wave comprising a carrier component at the incident frequency fi, and an acoustic-optical component scattered to one or the other of the acoustic sidebands of frequency fao=fa±n*fi.

Such methods are described in “Ultrasound-mediated optical tomography: a review of current methods” by Daniel S. Elson, Rug. Li, Christopher Dunsby, Robert Eckersley, and Meng-Xing Tang, published in Interface Focus (2011) vol. 1, pages 632-648.

In these known methods, one then obtains information about the medium by determining the weight of the acoustic-optical component relative to the carrier component. Furthermore, as the marked light wave comes from the focal spot of the ultrasonic wave, it is therefore possible to obtain information on the local light intensity in the scattering medium. In these methods, an image of the light intensity within the observation area is then formed, line by line, by sweeping the observation area with a series of focused ultrasonic waves.

These known methods have the disadvantage of requiring the emission of a large number of ultrasonic waves in order to obtain an image of the observation area, and are therefore slow. Furthermore, the signal-to-noise ratio is low and requires a large number of acquisitions at the same focal point to enable averaging them and obtaining a usable signal.

To this end, a first object of the invention is an acoustic-optical imaging method for imaging an observation area of a medium, the method comprising
an acquisition step during which at least one measurement signal associated with at least one non-focused acoustic wave is acquired,
   the acquisition step comprising at least one measurement operation wherein:
   a non-focused acoustic wave propagating in a propagation direction is generated in the observation area,
   an incident light wave is emitted in the observation area in order to generate a marked light wave comprising at least one acoustic-optical component shifted in frequency by the non-focused acoustic wave,
   a measurement signal containing information about the marked light wave is acquired; and
a processing step during which at least one value representative of a light intensity in the observation area is determined from the measurement signal.

In preferred embodiments of the invention, use may possibly be made of one or more of the following provisions:
the non-focused acoustic wave is an acoustic Plane wave;
the non-focused acoustic wave is an acoustic diverging wave;
the acquisition step includes a plurality of measurement operations such that, during the acquisition step, a plurality of measurement signals associated with a plurality of non-focused acoustic waves is acquired,

each non-focused acoustic wave being generated during a measurement operation f the plurality of measurement operations, the measurement signal associated with said non-focused acoustic wave being acquired during said measurement operation;
the non-focused acoustic waves of the plurality of non-focused acoustic waves propagate in non-collinear propagation directions;
the propagation directions of the plurality of non-focused acoustic waves cover a circular sector having a central angle greater than 10 degrees;
the propagation directions of the plurality of non-focused acoustic waves are separated by an angular spacing greater than 0.1 degrees, preferably greater than 0.5 degrees;
the propagation directions of the plurality of non-focused acoustic waves are separated by an angular spacing, of less than 45 degrees, preferably less than 20 degrees;
each measurement signal associated with a non-focused acoustic wave comprises a time series of light intensity values containing information relating to the acoustic-optical component of the marked light wave shifted in frequency by the non-focused acoustic wave;
each measurement signal associated with a non focused acoustic wave is sampled at a frequency greater than 2 MHz, preferably greater than 10 MHz;
the processing step comprises the implementation of an inverse Radon transform;
the processing step comprises the implementation of a beamforming algorithm;
the processing step comprises the implementation of a retroprojection or filtered retroprojection algorithm;
the processing step comprises the operations of:

determining a plurality of profile slices associated with at least one measurement signal, each profile slice being a function of a one-dimensional Fourier transform of an associated measurement signal,

determining a two-dimensional spectrum from the plurality of profile slices, and

determining at least one value representative of a light intensity in the observation area, said representative value being a function of a two-dimensional inverse Fourier transform of the two-dimensional spectrum;
the two-dimensional spectrum is determined by readjusting in a Fourier space the plurality of profile slices, preferably by readjusting each profile slice according to a propagation direction of a non-focused acoustic wave associated with the measurement signal associated with the profile slice;
a measurement operation is repeated n times in order to acquire n measurement signals associated with a propagation direction of a non-focused acoustic wave, and said n measurement signals are averaged together to determine a measurement signal associated with said non-focused acoustic wave used for the processing step;
the measurement operation is repeated a number n of times that is greater than or equal to 1.

The invention also relates to an acoustic-optical imaging system for imaging an observation area of a medium, the system comprising
an acquisition device for acquiring at least one measurement signal associated with at least one non-focused acoustic wave,

the acquisition device comprising:

a transducer array for generating, in the observation area, at least one non-focused acoustic wave propagating in a propagation direction,

a light-emitting device for emitting at least one incident light wave in the observation area, in order to generate at least one marked light wave comprising at least one acoustic-optical component shifted in frequency by said at least one non-focused acoustic wave,

a detector for acquiring at least one measurement signal representative of said at least one marked light wave; and
a processing device adapted to determine at least one value representative of a light intensity in the observation area, based on said at least one measurement signal acquired by the acquisition device.

Other features and advantages of the invention will be apparent from the following description of several of its embodiments given by way of non-limiting examples, with reference to the accompanying drawings.

In the drawings:

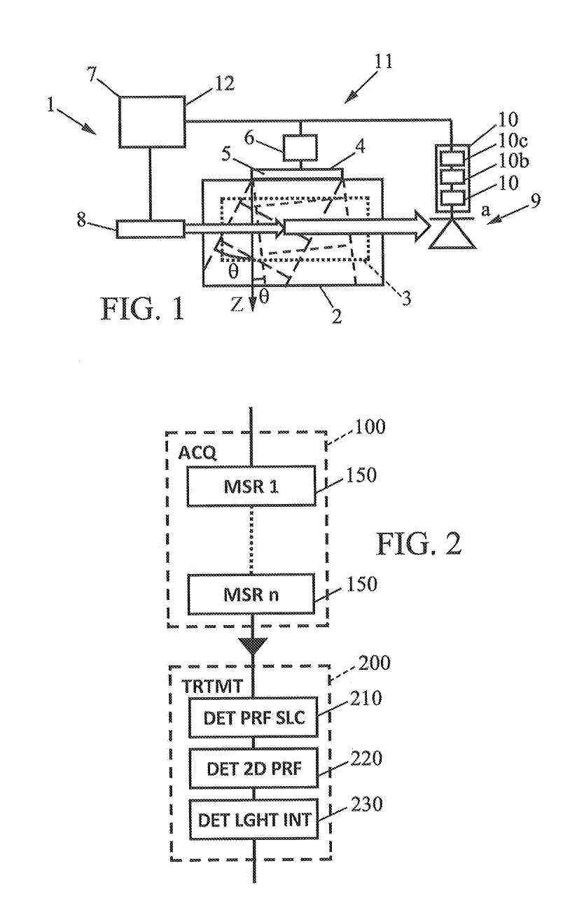

FIG. 1 is a schematic representation of a system according to an embodiment of the invention,

FIG. 2 is a schematic flowchart of a method according to an embodiment of the invention,

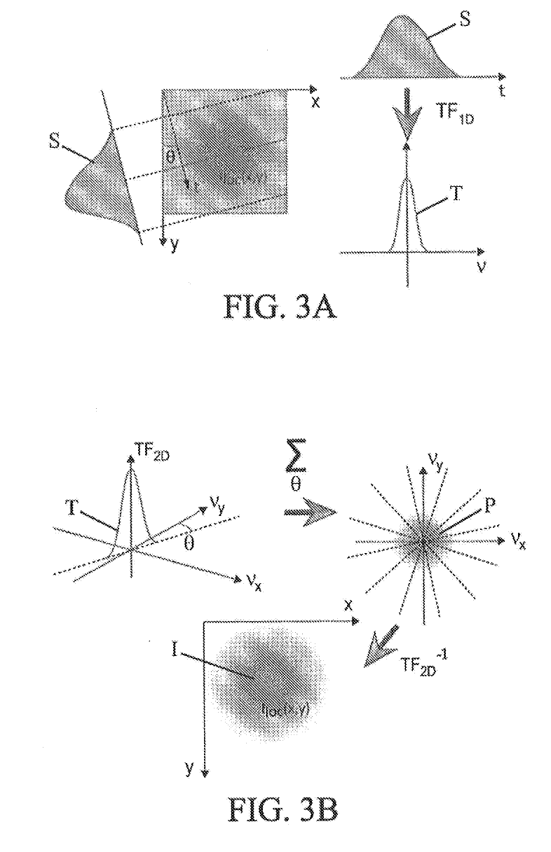

FIGS. 3A and 3B illustrate details of one embodiment of the operations in a processing step of the method of FIG. 2,

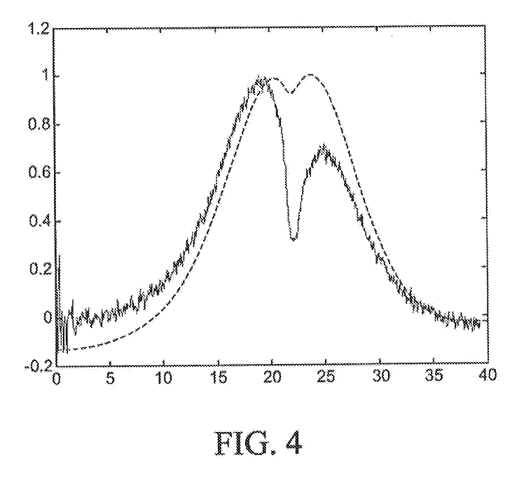

FIG. 4 shows representative light intensity values obtained with one embodiment of the invention and with a prior art method.

In the various figures, the same references designate identical or similar elements.

FIG. 1 schematically represents an acoustic-optical imaging system 1 according to one embodiment of the invention.

A medium 2 to be imaged is provided, for example an object or a biological tissue, therefore comprising an observation area 3. The observation area 3 may be on the surface of the medium 2 but may also be deep within the medium 2, for example several centimeters deep.

The medium 2 is a scattering medium. “Scattering medium” is understood to mean that beyond a typical thickness 1* (mean free path of transport), for example about a millimeter in biological media, information contained in a light wave traveling through the medium is completely scrambled and impossible to interpret without processing. This therefore renders conventional optical imaging impossible at depth. This phenomenon is also called multiple light scattering.

An acoustic transducer array 4 is in acoustic contact with the medium 2, either in direct contact, or for example acoustically coupled to the medium 2 by means of a coupling element such as a tank or a cushion filled with water.

The acoustic transducer array 4 is for example a linear array comprising for example several tens of transducers 5 (for example from 100 to 300). The transducers 5 are for example juxtaposed along an axis X. In alternative embodiments, the transducers 5 could be arranged to follow a curve, or arranged to form a two-dimensional array.

The acoustic transducer array 4 is controlled by control means which comprise for example an electronic rack 6 and a microcomputer 7 controlling the rack 6.

The acoustic transducer array 4 is thus able to generate, in the observation area 3, a non-focused acoustic wave propagating in a predefined propagation direction. The propagation direction can be controlled so as to generate, in the observation area 3, non-focused acoustic waves propagating in various propagation directions.

Without limitation, the acoustic transducer array 4 is for example capable of generating in the observation area 3 an ultrasonic wave having a central frequency of around a few megahertz, for example 8 MHz. The acoustic transducer array 4 is for example capable of generating in the observation area 3 a plurality of ultrasonic waves having selected propagation directions within a circular sector having a central angle greater than 30 degrees, for example 40 degrees.

In one embodiment of the invention, the non-focused acoustic waves are acoustic plane waves. In another embodiment, the non-focused acoustic waves are acoustic divergent waves.

The system 1 also comprises a light-emitting device 8. The light-emitting device 8 is capable of emitting, in the observation area 3, at least one incident light wave. In particular, the light-emitting device 8 is adapted to emit said light wave simultaneously with the emission of an ultrasonic wave by the acoustic transducer array 4. The light-emitting device 8 is for example a laser, or in general an emitting device which enables controlling the spectrum of the emitted incident light wave.

“Light wave” is understood in the broader sense, meaning electromagnetic radiation capable of propagating in the medium 2. In particular, it can be understood to mean electromagnetic radiation within the infrared, visible, or ultraviolet spectrum.

In an example provided by way of illustration and without limitation, the light-emitting device 8 is a single-frequency semiconductor laser amplified to 2 watts and 780 nanometers in wavelength (which therefore corresponds to an incident frequency fi). The polarization of the incident light wave can also be controlled. In some embodiments, the light wave may be temporally and spatially modulated or filtered before entering the medium 2.

The system 1 further comprises a detector 9 capable. of acquiring measurement signals representative of the marked light waves. The detector 9 is thus a photodetector sensitive to one or more electromagnetic wavelengths corresponding to wavelengths of the marked light wave. For example, the detector 9 is sensitive to an acoustic-optical component generated by an interaction between an incident light wave and a non-focused acoustic wave propagating in the observation area. The detector 9 may also be sensitive to a carrier component, meaning a component of the marked light wave at the incident frequency fi.

The detector 9 is for example a photodiode.

The system 1 may contain elements for pre-processing or post-processing the signal 10, possibly integrated into the detector 9. The elements for post-processing the signal 10 may for example include a high-pass filter 10a, a broadband amplifier 10b (for example Thorlabs, DHPVA), and an analog-to-digital converter 10c.

Thus in particular, the measurement signal can be sampled by the analog-to-digital converter 10e at a frequency greater than a few MHz, preferably greater than 10 megahertz, for example a sampling frequency of 40 MHz.

In this manner, each measurement Signal may in particular comprise a time series of light intensity values of an acoustic-optical component of a marked light wave shifted in frequency by a non-focused acoustic wave.

The transducer array 4, the light-emitting device 8, and the detector 9 may thus form an acquisition device 11 of a system 1 according to the invention. Such an acquisition device 11 is particularly suitable for acquiring a plurality of measurement signals associated with a plurality of non-focused acoustic waves, as will be detailed further below.

Finally, the system 1 according to the invention also comprises a processing device 12, adapted to determine at least one value representative of a light intensity in the observation area 3 based on the plurality of measurement signals acquired by the acquisition device 11.

An acoustic-optical imaging method for imaging an observation area of a medium is illustrated in more detail in FIG. 2 and can for example be implemented as follows, by means of the system 1.

During an acquisition step 100, a plurality of measurement signals S associated with a plurality of non-focused acoustic waves can be acquired.

Such an acquisition step 100 may include at least one measurement operation 150, enabling the acquisition of a measurement signal S associated with a non-focused acoustic wave, and in particular with a propagation direction of a non-focused acoustic wave θ.

Advantageously, the acquisition step 100 may include a plurality of measurement operations 150 so as to acquire a plurality of measurement signals (S) associated with a plurality of non-focused acoustic waves.

Thus, during each measurement operation 150:
a non-focused acoustic wave propagating in a propagation direction θ is generated in the observation area 3 by means of the transducer array 4,
an incident light wave is emitted in the observation area 3 in order to generate a marked light wave including at least one acoustic-optical component shifted in frequency by the non-focused acoustic wave by means of the light-emitting device 8, and
a measurement signal S representative of the marked light wave is acquired by means of the detector 9.

In particular, the plurality of measurement operations 150 of the acquisition step 100 may be such that the non-focused acoustic waves of the plurality of non-focused acoustic waves propagate in non-collinear propagation directions.

More specifically, the propagation directions θ of the plurality of non-focused acoustic waves may cover a circular sector having a central angle greater than 30 degrees. The circular sector May have a central angle greater than 60 degrees or 90 degrees, or even close to 180 degrees.

In one embodiment, the propagation directions θ of the plurality of non-focused acoustic waves are separated from each other by an angular spacing greater than 0.25 degrees, preferably greater than 0.5 degrees.

Moreover, the propagation directions θ of the plurality of non-focused acoustic waves may be separated by an angular spacing of less than 45 degrees, preferably less than 20 degrees.

Thus by way of non-limiting example, the acquisition step 100 may include the emission of plane waves in forty-one different propagation directions θ, said propagation directions having propagation angles between −20° and 20° relative to a direction Z normal to the transducer array 4, spaced apart by 1°. The plane waves may be emitted for example by a “Aixplorer” system from the company Supersonic Imagine.

In one embodiment of the invention, a measurement operation 150 in a propagation direction can be repeated n times to acquire n measurement signals S associated with a propagation direction θ of a non-focused acoustic wave. The measurement operation 150 may for example be repeated a number n of times greater than ten, for example one hundred times or one thousand times.

Such a repetition of the measurement operation 150 allows averaging together said n measurement signals so as to determine a measurement signal S to be used for the subsequent processing step. Such a measurement signal offers the advantage of having a higher signal-to-noise ratio.

The method next comprises a processing step 200 during which at least one value representative of a light intensity in the observation area is determined from the plurality of measurement signals.

Without limitation, this processing step 200 advantageously comprises the implementation of a Radon transform as illustrated in FIGS. 3A and 3B. The processing step may also comprise a double Fourier transform (temporal, then spatial).

Schematically, the processing step 200 may comprise the following operations;
determining (210) a plurality of profile slices associated with the plurality of measurement signals (FIG. 3A),
determining (220) a two-dimensional profile from the plurality of profile slices (FIG. 3B), and
determining (230) at least one value representative of a light intensity in the observation area, based on the two-dimensional profile (also FIG. 3B).

Specifically, we first determine a profile slice T for each measurement signal S obtained during the processing step.

To do so, a one-dimensional Fourier transform of the measurement signal S is used, which provides the associated profile slice T as shown in FIG. 1A. It is thus clear that the quality of the sampling of the measurement signal S is important to ensuring the preservation of information during the Fourier transform.

Then, from the plurality of profile slices T associated with the plurality of measurement signals S, a two-dimensional profile P is determined. As shown in FIG. 3B, the two-dimensional profile P is determined by readjusting in a Fourier space the plurality of profile slices. Each profile slice T is thus readjusted in the Fourier space according to the propagation direction θ of the non-focused acoustic wave which was associated with the measurement signal S associated with the profile slice T.

It is thus possible for example to readjust the profile slices T to fill the circular sector formed by the propagation directions of the non-focused waves.

Once the two-dimensional profile P is obtained, it is then possible to determine one or more value(s) representative of a light intensity I in the observation area 3, by a two-dimensional inverse Fourier transform of the two-dimensional profile P, as also illustrated in FIG. 3B.

In some embodiments of the invention, the profile slices T can be completed in order to determine the two-dimensional profile P.

FIG. 4 shows a result obtained during implementation of an exemplary embodiment of the invention. The graph of this figure illustrates the light intensity as a function of the position along an axis passing through the observation area. In this implementation of the invention, the imaged medium is an agar gel mixed with Intralipid 10%. Such a gel is highly scattering, such that beyond a millimeter, it is impossible to observe what is inside. An object that is absorbent, or locally more or less scattering than the gel, is placed in the scattering gel. As a purely illustrative example, a black electric wire sheath about 1 mm in diameter and 5 millimeter in length can be placed substantially in the middle of the gel in order to obtain the example in FIG. 4.

The graph of FIG. 4 compares the light intensity as a function of the position obtained by an implementation of the invention (dotted lines), with the light intensity as a function of the position obtained by an implementation of a known method as described in the introduction to the Present document, in which the observation area is scanned using focused ultrasonic waves (solid lines).

In both implementations, each measurement operation is repeated 1000 times to obtain an averaged measurement signal. In the implementation of the invention in FIG. 4 (dotted lines), plane waves between −20° and 20° spaced apart by 1° have been emitted by the transducer array 4

In the implementation of the known method of FIG. 4, the light intensity values are obtained by emitting a series of focused ultrasonic waves having their focal spots offset from one another by 0.2 mm so as to obtain an image of the light intensity in the observation area 3 with 100 columns.

As shown in FIG. 4, the signal-to-noise ratio (SNR) of the light intensity as a function of the position obtained by the implementation of the invention is much better than the signal-to-noise ratio of the light intensity as a function of the position obtained by the implementation of said known method.

This surprising result is explained by the fact that the non-focused ultrasonic waves enable encoding, in each marked light wave, the information coming from all points of the observation area. In contrast, in the known method, the use of focused ultrasonic waves implies that each marked light wave contains information coming only from the vicinity of the focal spot.

At equivalent signal-to-noise ratio, fewer measurement operations (tens of times fewer) are required with non-focused acoustic waves than with focused acoustic waves.

In comparison to the example presented, it is possible in variant embodiments to implement one or more of the following provisions:
use a more powerful laser,
use a lower noise photodiode, such as a Peltier-cooled photodiode,
use divergent waves,
scan a circular sector of greater central angle (360-degree CT scan)
use non-focused acoustic waves of higher power, for example close to the energy of standard ultrasound.

In this manner, at equivalent signal-to-noise ratio, it is possible to save imaging time by a factor of ten to a factor of one hundred or more, compared to the known methods described in the introduction of the present document.

1. An acoustic-optical imaging method for imaging an observation area of a medium, the method comprising:
an acquisition step during which a plurality of measurement signals, associated with a plurality of non-focused acoustic waves propagating in non collinear directions, is acquired,
the acquisition step comprising a plurality of measurement operations such that, in each measurement operation of the plurality of measurement operations (150);
a non-focused acoustic wave propagating in a propagation direction is generated in the observation area,
an incident light wave is emitted in the observation area in order to generate a marked light wave comprising at least one acoustic-optical component shifted in frequency by the non-focused acoustic wave,
a measurement signal, associated with said non-focused acoustic wave, containing information about the marked light wave is acquired; and
a processing step during which at least one value representative of a light intensity in the observation area is determined from said plurality of measurement signals.

2. A method according to claim 1, wherein the non-focused acoustic wave is an acoustic plane wave.

3. The method according to claim 1, wherein the non-focused acoustic wave is an acoustic diverging wave.

4. (canceled)

5. (canceled)

4. The method according to claim 1, wherein the propagation directions of the plurality of non-focused acoustic waves cover a circular sector having a central angle greater than 10 degrees.

5. The method according to claim 1, wherein the propagation directions of the plurality of non-focused acoustic waves are separated by an angular spacing greater than 0.1 degrees, preferably greater than 0.5 degrees.

6. The method according to claim 1, wherein the propagation directions of the plurality of non-focused acoustic waves are separated by an angular spacing of less than 45 degrees, preferably less than 20 degrees.

7. The method according to claim 1, wherein each measurement signal associated with a non-focused acoustic wave comprises a time series of light intensity values containing information relating to the acoustic-optical component of the marked light wave shifted in frequency by the non-focused acoustic wave.

8. The method according to claim 1, wherein each measurement signal associated with a non-focused acoustic wave is sampled at a frequency greater than 2 MHz, preferably greater than 30 megahertz.

9. The method according to claim 1, wherein the processing step comprises the implementation of an inverse Radon transform.

10. The method according to claim 1, wherein the processing step comprises the implementation of a beamforming algorithm.

11. The method according to claim 1, wherein the processing step comprises the implementation of a retroprojection or filtered retroprojection algorithm.

12. The method according to claim 1, wherein the processing step comprises the operations of:
determining a plurality of profile slices associated with at least one measurement signal, each profile slice being a function of a one-dimensional Fourier transform of an associated measurement signal,
determining a two-dimensional spectrum from the plurality of profile slices, and
determining at least one value representative of a light intensity in the observation area, said representative value being a function of a two-dimensional inverse Fourier transform of the two-dimensional spectrum.

13. The method according to claim 12, wherein the two-dimensional spectrum is determined by readjusting in a Fourier space the plurality of profile slices, preferably by readjusting each profile slice according to a propagation direction of a non-focused acoustic wave associated with the measurement signal associated with the profile slice.

14. The method according to claim 1, wherein a measurement operation is repeated n times in order to acquire n measurement signals associated with a propagation direction of a non-focused acoustic wave, and wherein said n measurement signals are averaged together to determine a measurement signal associated with said non-focused acoustic wave used for the processing step.

15. The method according to claim 1, wherein the measurement operation is repeated a number n of times that is greater than or equal to 1.

16. The acoustic-optical imaging system for imaging an observation area of a medium, the system comprising
an acquisition device for acquiring a plurality of measurement signals associated with a plurality of non-focused acoustic waves propagating in non collinear directions,
the acquisition device comprising:
a transducer array for generating, in the observation area, a plurality of non focused acoustic waves propagating in non collinear directions,
a light-emitting device for emitting at least one incident light wave in the observation area, in order to generate a plurality of marked light waves comprising at least one acoustic-optical component shifted in frequency by said at least one non-focused acoustic wave,
a detector for acquiring a plurality of measurement signals associated with said plurality of non-focused acoustic waves and respectively representative of each marked light wave of the plurality of non-focused acoustic waves; and
a processing device adapted to determine at least one value representative of a light intensity in the observation area, based on said plurality of measurement signals acquired by the acquisition device.
